(19) United States
(12) Patent Application Publication
(10) Pub. No.: US 2026/0137499 A1
(43) Pub. Date: May 21, 2026

(54) OCCLUSIVE CUFF AND IMPLANTABLE OCCLUSIVE SYSTEM COMPRISING SUCH A CUFF

(71) Applicant: UROMEMS, Grenoble (FR)

(72) Inventors: Hamid LAMRAOUI, Grenoble (FR); Christopher MASRI, Grenoble (FR); John MURPHY, Meylan (FR)

(21) Appl. No.: 19/447,260

(22) Filed: Jan. 13, 2026

Related U.S. Application Data
(63) Continuation-in-part of application No. 17/836,322, filed on Jun. 9, 2022, now Pat. No. 12,551,324.
Application No. 17/836,322 is a continuation of application No. 16/767,037, now abandoned.

(30) Foreign Application Priority Data
Nov. 27, 2018 (FR) 1761287

(51) Int. Cl. A61F 2/00

(52) U.S. Cl. CPC A61F 2/004; A61B 5/02233; A61B 17/135

(57) ABSTRACT

The invention relates to an occlusive cuff comprising:
a band configured to be wound around an anatomical duct according to a fixed [OR constant] circumference;
an unstretched inflatable pouch on an inner face of the band, configured to be fluidically connected to an external fluid container;
the unstretched inflatable pouch extending over less than or equal to 75% of the circumference;
the occlusive cuff being operable between
   an occluding configuration wherein the inflatable pouch is filled with fluid to exert a compression onto the anatomical duct for occluding the anatomical duct without pinching the anatomical duct, and
   a released configuration wherein the inflatable pouch is deflated to release the compression onto the anatomical duct and at least partially open the anatomical duct.

### FIELD OF THE INVENTION

The present invention relates to an occlusion cuff intended to obstruct selectively an anatomical duct, such as a urethra or a bladder neck, as well as an implantable occlusion system comprising such a cuff.

### PRIOR ART

Implantable occlusion systems exist intended to selectively obstruct an anatomical duct, for example to remedy incontinence (case of artificial urinary or anal sphincters) or to limit intake of food into the stomach (case of gastric bands). The obstruction of the anatomical duct is brought about by the compression exerted by a cuff wound around said duct.

Among cuffs, the present text focuses on those based on fluid technology (the cuff comprising an inflatable reservoir coupled to a fluid circuit making it possible to fill or to empty selectively said reservoir, according to the compression to exert on the anatomical duct).

Among occlusion systems, some are manual, that is to say that it is a user (for example the patient himself) who controls the compression applied by the cuff. Other systems are automatic, that is to say that they comprise one or more sensors, an actuator and a control unit for controlling the actuator to impose a determined compression of the anatomical duct by the cuff, without any intervention of a user being necessary.

The document WO 2016/083428 describes an automatic implantable occlusion system. This system comprises a fluid circuit comprising:
an inflatable occlusion cuff containing a variable volume of fluid, intended to surround at least a part of a natural duct to occlude,
a recipient of variable volume 41 filled with a fluid, said recipient comprising a fixed part 42 and a movable part 43,
fluid connection between the recipient and the occlusion cuff.

Furthermore, this system comprises an actuator mechanically coupled with the movable part of the recipient so as to displace said movable part linearly with respect to the fixed part to adjust the volume of the recipient.

In the following description and the claims, the recipient may also be referred to as a container.

The actuator and the recipient of variable volume are arranged in a leak tight housing containing a gas.

FIGS. 1A and 1B are views of an occlusion cuff 1′ that can be used in this system, respectively in the free state (before implantation), and once implanted around the anatomical duct 2 to obstruct.

Said cuff comprises a band 10 suited to surrounding said anatomical duct, and an inflatable reservoir 11′ arranged on one face of the band 10, substantially over the entire length thereof. The inflatable reservoir 11 comprises at one of its ends a connector 12 making it possible to ensure a fluid connection between the inside of the reservoir 11 and a tubing 3 connected to the recipient.

At its end opposite to said connector 12, the band has an oblong opening 14, which is intended to be engaged on the connector 12 in order to maintain the cuff in position wound around the anatomical duct 2.

In the present text, “length” of the cuff is taken to mean the inner circumference of the cuff when said cuff is in its closed position. This length is noted L₁₀ in FIG. 2A. According to the closing device of the cuff, this length is thus generally less than the total length of the band.

Cuffs of different lengths exist, according to the targeted applications and patients.

The length of the occlusion cuff is chosen by the surgeon, as a function of the circumference of the anatomical duct at the location planned for the cuff.

In the case of artificial urinary sphincters, the cuff is generally implanted in women around the bladder neck, which has a considerable circumference. Typically, the length of the cuff for such an application is of the order to ten or so centimetres.

However, the use of a cuff of this dimension poses a certain number of problems for the implantation of the system and the operation thereof.

A first drawback of such a cuff is its bulk. Yet, the implantation of such a cuff around the bladder neck of women is awkward because there is little space around the bladder neck to insert the cuff. Moreover, the location of the cuff is very close to the vaginal wall, which the surgeon must take care not to perforate during the implantation.

On the one hand, the volume of the cuff being important, the fluid recipient must be dimensioned as a consequence to procure sufficient compression of the anatomical duct by the cuff. This implies an increase in the bulk of the housing containing said recipient.

Furthermore, the variation in volume of the recipient, which is implemented with a considerable amplitude, also leads to a reduction in the autonomy of the energy source which supplies the actuator, when said actuator is also arranged in the housing.

Consequently, the use of a cuff of large dimension implies at one and the same time great complexity of the implantation operation, an increase in the volume of the implantable housing and a decrease in the autonomy of the system.

### DESCRIPTION OF THE INVENTION

An aim of the invention is thus to design an occlusion cuff that can be easily implanted around an anatomical duct having an important circumference, without causing the aforementioned problems.

To this end, the invention proposes an occlusive cuff comprising:
a band configured to be wound around an anatomical duct according to a fixed circumference;
an unstretched inflatable pouch on an inner face of the band, configured to be fluidically connected to an external fluid container;
the unstretched inflatable pouch extending over less than or equal to 75% of the circumference;
the occlusive cuff being operable between
   an occluding configuration wherein the inflatable pouch is filled with fluid to exert a compression onto the anatomical duct for occluding the anatomical duct without pinching the anatomical duct, and
   a released configuration wherein the inflatable pouch is deflated to release the compression onto the anatomical duct and at least partially open the anatomical duct.

In accordance with its common acceptance, the term “free” signifies in the present text that the inner surface of the band does not support any additional element, which could be capable of being interposed between the band and the anatomical duct.

In the present text, the term “inner” designates the face of the cuff which, when the cuff is in closed position, is intended to come into direct contact with the anatomical duct. The term “outer” designates the opposite face.

The occlusive cuff may further comprise at least one protrusion extending from the inner face of the band over at least 10% of the circumference.

Advantageously, a width of at least one protrusion is between 10 and 100% of a width of the inflatable pouch.

Preferably, a height of at least one protrusion is between 10 and 100% of a height of the inflatable pouch in the occluding configuration.

The occlusive cuff may comprise at least two protrusions extending from the inner face of the band, wherein the protrusions together extend over at least 15% of the circumference.

In certain embodiments, the at least one protrusion is a solid block made from a biocompatible elastomer.

In other embodiments, the at least one protrusion is an elastomeric compartment filled with a gel.

Preferably, the at least one protrusion is adjacent to the inflatable pouch.

A length of the inflatable pouch may be chosen such that when the band is wound upon itself around the anatomical duct and when the pouch is inflated, the anatomical duct is compressed between the wall of the pouch and a free inner surface of the band adjacent to the occlusive cuff, the pouch exerting a push force of the anatomical duct against said free inner surface of the band.

Advantageously, the inflatable pouch and the inner surface of the band not covered by the inflatable pouch are configured to be in direct contact with the anatomical duct when the band is wound around said anatomical duct.

Preferably, the band is formed of a textile coated with a biocompatible elastomer.

The inflatable pouch may be made of a biocompatible elastomer.

Preferably, said biocompatible elastomer is silicone.

The occlusive cuff may further comprise a connector arranged at one end of the band to ensure a fluid connection between the pouch and a tubing connecting the inflatable pouch and the external container.

Advantageously, the occlusive cuff further comprises a closing device configured to maintain the band wound around the anatomical duct, wherein the closing device comprises an opening formed at one end of the band and able to engage around the connector.

The occlusive cuff may further comprise a closing device configured to maintain the band wound around the anatomical duct, wherein the closing device comprises a surface provided with teeth at one end of the band and an attachment device comprising a slot for the insertion of said end, configured to block the teeth against an extraction force of said end.

The invention further relates to an implantable occlusive system comprising:
a housing comprising a fluid container and a pump; and
an occlusive cuff comprising:
   a band configured to be wound around an anatomical duct according to a fixed circumference;
   an unstretched inflatable pouch on an inner face of the band, in fluidic communication with the fluid container;
   the unstretched inflatable pouch extending over less than 75% of the circumference;
   the occlusive cuff being operable between
      an occluding configuration wherein the inflatable pouch is filled with fluid to exert a compression onto the anatomical duct for occluding the anatomical duct without pinching the anatomical duct, and
      a released configuration wherein the inflatable pouch is deflated to release the compression onto the anatomical duct and at least partially open the anatomical duct; and
   wherein the band is remote from the housing, the pump being configured to transfer fluid between the fluid container and the inflatable pouch to selectively increase or decrease a volume of fluid in the pouch.

According to an application of the invention, the system consists of an artificial urinary sphincter.

An occlusion cuff for selectively obstructing an anatomical duct, may comprise:
a band suited to surrounding said anatomical duct, provided with a closing device suited to maintaining the band wound upon itself over a determined length, and
an inflatable reservoir arranged on an inner face of the band.

Said cuff is characterised in that the inflatable reservoir extends over only a part of the length of the band, another part of said length defining a free inner surface of the band configured to form a bearing zone for the anatomical duct, such that when the band is maintained wound upon itself by the closing device, the inflatable reservoir is opposite said free inner surface of the band.

In a particularly advantageous manner, the length of the reservoir is chosen such that when the band is wound upon itself around the anatomical duct and when the reservoir is inflated, the anatomical duct is compressed between the wall of the reservoir and the free surface of the band, the reservoir exerting a push force of the anatomical duct against said free surface of the band.

The inflatable reservoir preferably extends over a length less than or equal to 75% of the length of the band.

The length of the band may be comprised between 30 and 110 mm, preferably between 70 and 110 mm.

According to an embodiment, the band is formed of a textile coated with a biocompatible elastomer. The inflatable reservoir may be made of a biocompatible elastomer. According to an embodiment, said biocompatible elastomer is silicone.

Advantageously, the cuff further comprises a connector arranged at one end of the band to ensure a fluid connection between the reservoir and a tubing.

According to an embodiment, the closing device comprises an opening formed at one end of the band and able to engage around the connector to maintain the band wound upon itself.

Alternatively, the closing device comprises a surface provided with notches at one end of the band and an attachment device comprising a slot for the insertion of said end, configured to block the notches against an extraction force of said end. Preferably, these notches are arranged on the outer face of the band.

The invention also relates to an implantable occlusion system comprising a cuff such as described above and an implantable housing comprising a pump in fluid connection with the cuff to vary the volume of fluid in the inflatable reservoir.

According to a particular application of the invention, the occlusion system is an artificial urinary sphincter.

### DESCRIPTION OF THE FIGURES

Other characteristics and advantages of the invention will become clear from the detailed description that follows, with reference to the appended drawings among which:

FIG. 1A is a schematic view of an occlusion cuff of known type, such as supplied to the surgeon with a view to implantation;

FIG. 1B is a schematic view of the cuff of FIG. 1A, after implantation around the anatomical duct (represented as not exerting any compression on said duct);

FIG. 2A is a schematic view of an occlusion cuff according to an embodiment of the invention, such as supplied to the surgeon with a view to implantation;

FIG. 2B a schematic view of the cuff of FIG. 2A, after implantation around the anatomical duct (represented as not exerting any compression on said duct);

FIG. 2C is a schematic view of the cuff of FIG. 2A, after implantation around the anatomical duct, in configuration of obturation of said duct;

FIG. 3 illustrates an alternative of the system for closing the occlusion cuff;

FIG. 4A illustrates a simulation by finite elements of the compressive stress generated in a bladder neck by the compression exerted by a cuff such as illustrated in FIG. 1A;

FIG. 4B illustrates a simulation by finite elements of the compressive stress generated in the bladder neck by the compression exerted by a cuff according to an embodiment of the invention;

FIG. 5 illustrates an implantable occlusion system comprising a cuff according to an embodiment of the invention.

FIG. 6 is a schematic view of another embodiment of an occlusive cuff, such as supplied to the surgeon with a view to implantation.

FIG. 7 illustrates an embodiment of an occlusive cuff having a protrusion being a solid block of a biocompatible elastomer.

FIG. 8 illustrates an embodiment of an occlusive cuff having a protrusion which is an elastomeric compartment filled with a gel.

Identical reference signs from one figure to the other designate elements that are identical or at least fulfil the same function.

### DETAILED DESCRIPTION OF EMBODIMENTS OF THE INVENTION

Compared to known occlusion cuffs, the invention proposes reducing the length of the inflatable reservoir. In the following description and the claims, the inflatable reservoir may also be referred to as an inflatable pouch. FIGS. 2A-2C schematically illustrate a cuff 1 according to an embodiment of the invention, respectively in the free state, in the closed state without exerting compression on the duct and in a configuration of obstruction of said duct.

The inflatable reservoir 11 extends over less than 75% of the length L₁₀ of the band, preferably less than 60% of the length of the band, and in an even more preferred manner less than 50% of the length of the band. The length of the inflatable reservoir is noted L₁₁.

Thus, for example, for a cuff 10 cm long, the length of the inflatable reservoir is 4.5 cm.

In other words, the inner face of the band 10 is broken down into a region of length L₁₁ on which is arranged the reservoir 11, and a region of length L₁₃ which forms a free surface, that is to say devoid of reservoir, and which constitutes a bearing zone for the anatomical duct.

When the cuff is in closed position, the wall of the reservoir 11 thus lies facing the free surface 13 of the band (the anatomical duct 2 being interposed between these two surfaces, as shown in FIG. 2B).

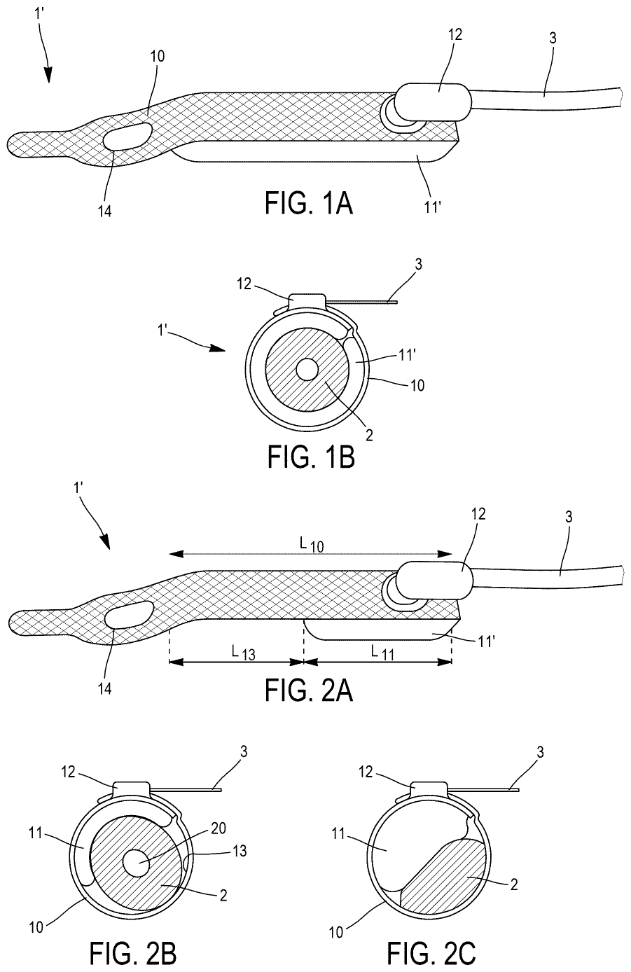

Conversely, in a cuff of known type such as represented in FIGS. 1A and 1B, the two ends of the reservoir are flush when the cuff is closed. Even if a slight play may subsist between these two ends, notably to take account of the variation in volume of the reservoir when it is inflated, the corresponding zone of the band does not constitute a bearing zone for the anatomical duct.

Returning to the invention, when the reservoir 11 is inflated, it pushes the anatomical duct against the free surface 13 of the band, thus progressively obstructing the duct (cf. FIG. 2C). Thus, the anatomical duct is compressed between, on the one hand, the reservoir 11 and, on the other hand, the free surface 13.

Advantageously, a panoply of cuffs of different lengths is made available to the surgeon, from which the surgeon will chose the most suitable for the patient and for the location planned for the implantation. In this panoply, the length of the band is generally comprised between 3 and 11 cm.

For cuffs of small length (that is to say typically less than 7 cm), the inflatable reservoir may be arranged over the entire length of the cuff.

From a certain length of cuff (for example 7 cm), the length of the inflatable reservoir may be fixed (for example of the order of 4.5 cm). Thus, it is possible to use the same pump for all cuff dimensions, which avoids multiplying the housing references.

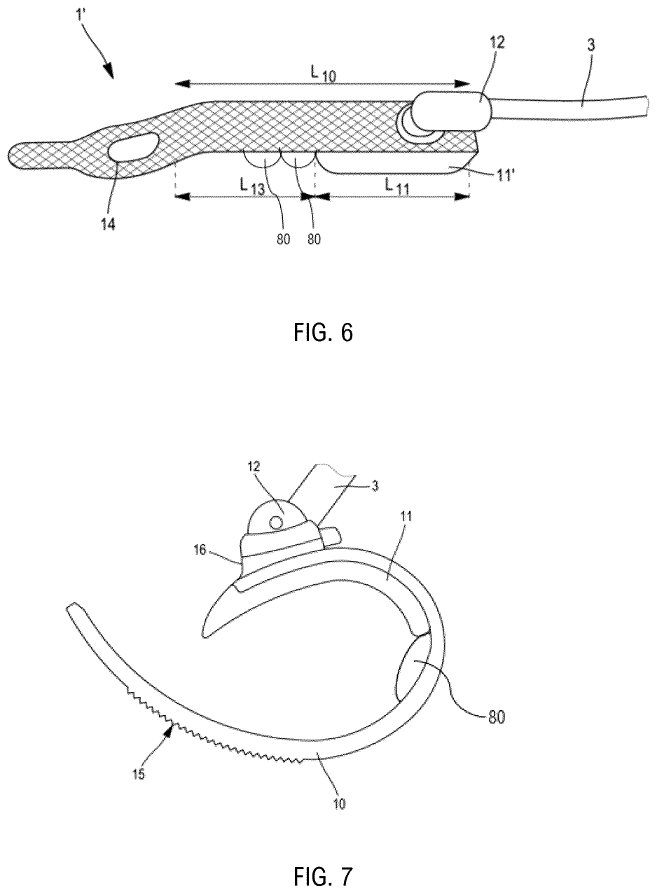

In certain embodiments, as illustrated in FIG. 6 and in FIG. 7, the cuff further comprises one or several protrusions extending from the inner face of the band.

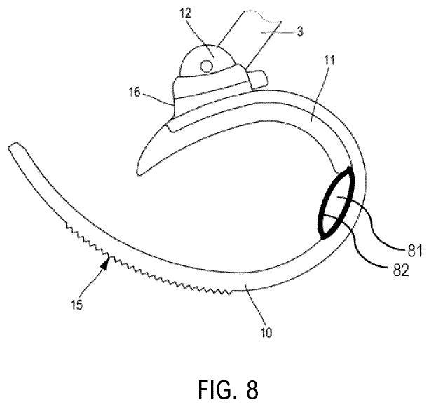

As shown in FIG. 7, a protrusion may be a solid block, preferably made from a biocompatible elastomer. Alternatively, as illustrated in FIG. 8, one or more protrusions may comprise an elastomeric compartment 82, which is filled with a gel 81.

Advantageously, the protrusions have a smooth rounded form. A protrusion may for example be dome shaped. As an alternative, a protrusion may have a flat upper surface and curved edges. Such rounded shapes allow for preventing injury of the anatomical duct by the protrusions.

The protrusions are preferably arranged in a position adjacent to the inflatable reservoir, such that the inflatable reservoir and at least one protrusion are in contact with each other when the band is wound around an anatomical duct and the reservoir is at least partially inflated. Hereby, the protrusions allow further compression of the anatomical duct without preventing the free inner surface of the band from supporting the anatomical duct.

The protrusions cover a portion of the inner face of the band which preferably extends over at least 10% of the circumference. This portion may be covered by a single protrusion, or by a plurality of protrusions which may be adjacent to each other. In the case of two or more protrusions, the plurality of protrusions may for example cover at least 15% or more of the total circumference of the band, including the length of the inflatable pouch.

When several protrusions are arranged on a single band, these protrusions may be made of identical or different materials and may either be of similar length and width or have different dimensions relative to each other.

The height of each protrusion is typically equal to or smaller than the height of the inflatable pouch in the inflated state. Hence, the height of such a protrusion may be comprised between 10 and 100% of the height of the inflatable pouch in the inflated state.

Similarly, the width of each protrusion is preferably equal to or smaller than the width of the inflatable pouch. The width of a protrusion may for example be comprised between 10% and 100% of a width of the inflatable pouch. The presence of one or more protrusions may improve the compression of the anatomical duct. The cuff may be manufactured in the following manner.

On the one hand, the band is formed of a textile coated with a biocompatible elastomer. Advantageously, the textile is a biocompatible polyester fabric, known notably by the name of DACRON™ and the elastomer is silicone. The band may be cut, for example by water jet, from a sheet of large dimensions. The band thus formed has the advantage of being smooth, of not setting into the anatomical tissues (thus allowing potential explanation of the cuff) and of not being stretchable.

On the other hand, the reservoir is made of a biocompatible elastomer, for example silicone, preferably by dipping so as to ensure a perfectly smooth surface state in order to ensure a flexible contact with the anatomical duct.

The reservoir is bonded onto the band.

Optionally, one or more protrusions may be bonded onto the band, before or after the bonding of the reservoir. If the protrusions comprise a filling such as a gel, the filling may be introduced into a compartment before the bonding of the compartment onto the band. Alternatively, the compartment may be bonded onto the band and be filled at a later stage.

The connector is for its part moulded and bonded onto the face of the band opposite to the reservoir while passing through said band to ensure a fluid connection between the reservoir and the tubing which is sleeved on the connector.

According to an embodiment, illustrated in FIG. 2A, the closing device of the cuff 1 comprises an oblong opening 14 intended to engage on the connector 12.

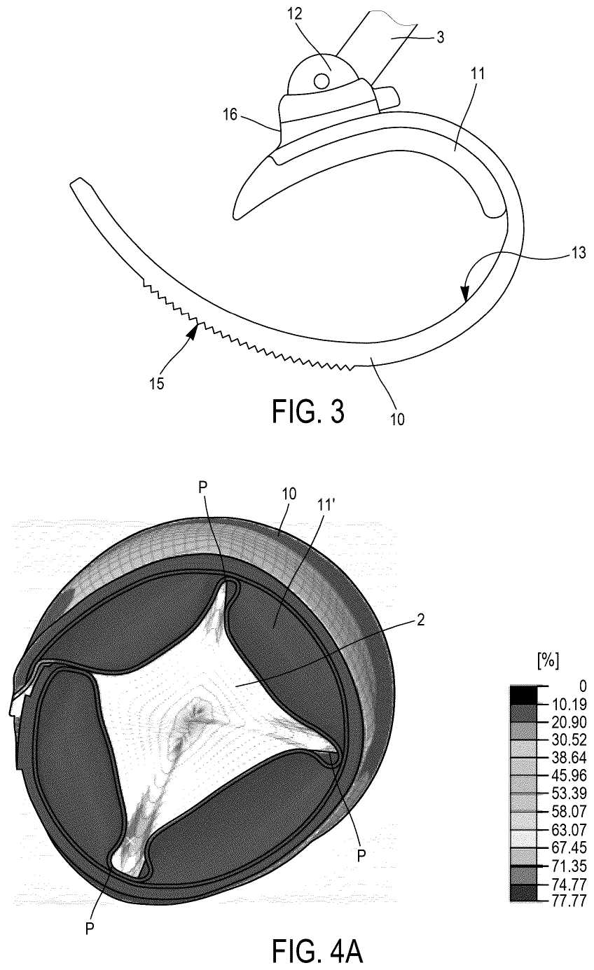

According to another embodiment, illustrated in FIG. 3, the band has at a first end a plurality of notches 15 and at its other end, preferably at the level of the connector 12, an attachment device 16 comprising a slot suited to enabling the insertion of the first end and provided with a retaining system only allowing a displacement of the band 10 in the direction of the insertion (the notches opposing a traction movement aiming to make the band come out of the slot). Advantageously, the notches 15 lie on the outer face of the band 10, in order not to risk eroding the compressed part of the duct. The notches may form an integral part of the band or be manufactured by moulding then bonded onto the band.

Naturally, those skilled in the art could choose any other closing device without however going beyond the scope of the present invention.

The reduction in the length of the inflatable reservoir has three main advantageous effects.

A first effect is a decrease in the bulk of the cuff, which facilitates its implantation in zones where the available space is limited. In the deflated state prior to implantation, the cuff is in the form of a band having a small thickness, which can thus be inserted relatively easily into the zone dissected around the bladder neck, unlike the cuff described in the document WO 2013/165563, which uses the cooperation of an anvil integral with the inner surface of the band and a hammer integral with the reservoir to compress the anatomical duct. The anvil and hammer being relatively rigid, they have a non-negligible bulk in the implantation zone. Conversely, in the present invention, as is clearly shown in FIGS. 2B and 2C, when the cuff is implanted around the anatomical duct, said duct is in direct contact with the reservoir 11 and with the free surface 13 of the band which are flexible parts, which makes it possible to minimise the thickness of the cuff and reduce the risk of erosion of body tissues.

A second advantageous effect of the invention is a reduction in the volume of fluid necessary to swell the reservoir in order to obstruct the anatomical duct. In the presence of one or more protrusions, the anatomical duct is partly compressed by the protrusions. Hence, the volume of the reservoir may be reduced. Consequently, the volume of the fluid required for swelling the reservoir is further reduced with respect to the embodiment without protrusions.

Consequently, the volume of the recipient of variable volume contained in the housing remains limited and may be further limited in case of one or more protrusions in the inner face of the band, and the autonomy of the energy source is not penalised.

A third effect of the invention concerns the handling of cuffs having different lengths. A plurality of cuffs of having different lengths may be made available for preparing the implantation of a cuff into a patient, in order to choose the most suitable length during the intervention. In this case, bands of different lengths, preferably all available lengths, may comprise inflatable pouches of identical size and additionally have protrusions depending on the length of the band. The number and/or the size of the protrusions may be adapted to the length of each cuff in order to optimize the compression of the anatomical duct without need for different types of pouches and different amounts of fluids required for inflating these pouches. For example, a short band may have an inflatable pouch only and no protrusion, a medium size band may have an identical pouch and single protrusion, and a long band may have another identical pouch and two protrusions aligned along the length of the band.

In this way, the pouches require the same amount of fluid for inflating. Hence, the volume of the fluid to be provided for inflating each cuff, and the fluid container of an automatic implantable occlusion system are independent of the chosen length of the cuff. This simplifies the handling of different cuff sizes, as all available cuffs may be used in combination with the same fluid container and the same amount of fluid.

A fourth effect is a modification of the mode of action of the cuff on the anatomical duct.

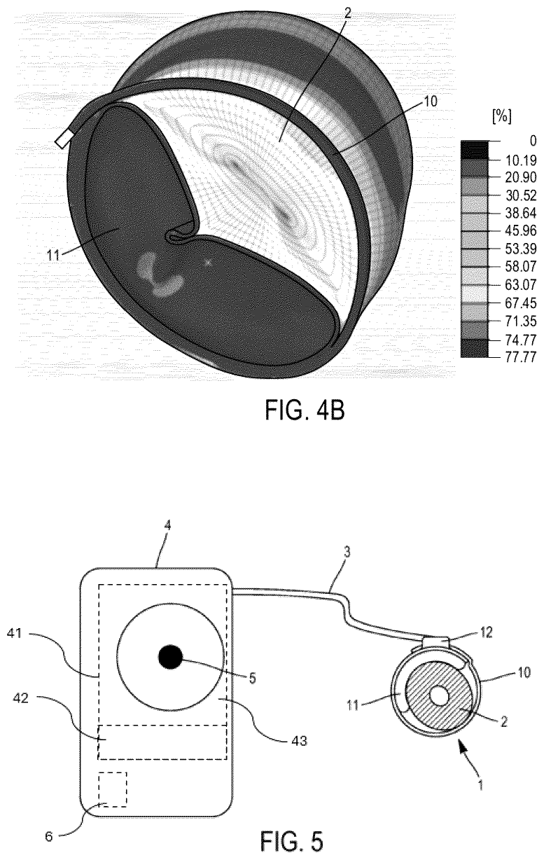

FIG. 4A shows the result of a simulation by finite elements of the compressive stresses applied to the bladder neck by a cuff according to the prior art, such as illustrated in FIG. 1A, for a fluid pressure in the cuff of 120 cm H2O. It may be observed that the curvature of the cuff combined with a swelling of the reservoir has the effect of dividing the reservoir 11′ into four pockets which each extend over around one quarter of the inner circumference of the cuff. Yet, the bladder neck is pinched between two adjacent pockets, and is subjected, in these pinch zones P, to local mechanical stresses well above the stresses to which the remainder of the neck is subjected. These localised stresses are liable to damage the bladder neck, for example atrophying it.

In FIGS. 4A and 4B, the stress levels represented by dark colours correspond to high stresses on the colour scale shown on the right of each simulation.

FIG. 4B shows the result of a simulation by finite elements of the compressive stresses applied to the bladder neck by a cuff according to an embodiment of the invention, such as illustrated in FIG. 2A, for a fluid pressure in the cuff of 120 cm H₂O. It may be observed that the reduction in the length of the inflatable reservoir 11 has the effect of eliminating the pinch zones of the bladder neck, and consequently eliminating the excess stresses undergone locally by the bladder neck. The bladder neck is thus less likely to be damaged.

FIG. 5 illustrates an implantable occlusion system comprising an implantable housing comprising a pump, and an occlusion cuff such as described above, in fluid connection with the pump. The pump is advantageously one of the devices described in the document WO 2016/083428.

The housing 4 contains the recipient of variable volume, the actuator as well as said electronic module(s) and, if need be, the energy source. The housing contains a gas, for example air. Said housing must be leak tight to avoid any transfer of fluid or gas from or to the intracorporal medium. The housing is made of a biocompatible material and may for example be made of implantable titanium and sealed by laser welding. A control of the leak tightness may notably be carried out with helium (for example, leakage rate less than 10⁻⁹ mbar. l/s of helium) to ensure the total leak tightness of the housing for the period for which the system is implanted.

The recipient of variable volume is connected to the cuff 1 by the tubing 3.

Advantageously, the housing comprises, in a wall delimiting the recipient of variable volume, a puncture port 5 perforable by a needle and able to close in a leak tight manner after removal of the needle, making it possible to inject or to remove fluid from the recipient.

### REFERENCES

WO 2016/083428

WO 2013/165563

1. An occlusive cuff comprising:
a band configured to be wound around an anatomical duct according to a fixed circumference;
an unstretched inflatable pouch on an inner face of the band, configured to be fluidically connected to an external fluid container;
the unstretched inflatable pouch extending over less than or equal to 75% of the circumference;
the occlusive cuff being operable between
an occluding configuration wherein the inflatable pouch is filled with fluid to exert a compression onto the anatomical duct for occluding the anatomical duct without pinching the anatomical duct, and
a released configuration wherein the inflatable pouch is deflated to release the compression onto the anatomical duct and at least partially open the anatomical duct.

2. The occlusive cuff of claim 1, further comprising at least one protrusion extending from the inner face of the band over at least 10% of the circumference.

3. The occlusive cuff of claim 2, wherein a width of at least one protrusion is between 10 and 100% of a width of the inflatable pouch.

4. The occlusive cuff of claim 2, wherein a height of at least one protrusion is between 10 and 100% of a height of the inflatable pouch in the occluding configuration.

5. The occlusive cuff of claim 2, comprising at least two protrusions extending from the inner face of the band, wherein the protrusions together extend over at least 15% of the circumference.

6. The occlusive cuff of claim 2, wherein the at least one protrusion is a solid block made from a biocompatible elastomer.

7. The occlusive cuff of claim 2, wherein the at least one protrusion is an elastomeric compartment filled with a gel.

8. The occlusive cuff of claim 2, wherein the at least one protrusion is adjacent to the inflatable pouch.

9. The occlusive cuff of claim 1, wherein a length of the inflatable pouch is chosen such that when the band is wound upon itself around the anatomical duct and when the pouch is inflated, the anatomical duct is compressed between the wall of the pouch and a free inner surface of the band adjacent to the occlusive cuff, the pouch exerting a push force of the anatomical duct against said free inner surface of the band.

10. The occlusive cuff of claim 1, wherein the inflatable pouch and the inner surface of the band not covered by the inflatable pouch are configured to be in direct contact with the anatomical duct when the band is wound around said anatomical duct.

11. The occlusive cuff of claim 1, wherein the band is formed of a textile coated with a biocompatible elastomer.

12. The occlusive cuff of claim 1, wherein the inflatable pouch is made of a biocompatible elastomer.

13. The cuff of claim 12, wherein said biocompatible elastomer is silicone.

14. The occlusive cuff of claim 1, further comprising a connector arranged at one end of the band to ensure a fluid connection between the pouch and a tubing connecting the inflatable pouch and the external container.

15. The occlusive cuff of claim 14, further comprising a closing device configured to maintain the band wound around the anatomical duct, wherein the closing device comprises an opening formed at one end of the band and able to engage around the connector.

16. The occlusive cuff of claim 1, further comprising a closing device configured to maintain the band wound around the anatomical duct, wherein the closing device comprises a surface provided with teeth at one end of the band and an attachment device comprising a slot for the insertion of said end, configured to block the teeth against an extraction force of said end.

17. An implantable occlusive system comprising:
a housing comprising a fluid container and a pump; and
an occlusive cuff comprising:
a band configured to be wound around an anatomical duct according to a fixed circumference;
an unstretched inflatable pouch on an inner face of the band, in fluidic communication with the fluid container;
the unstretched inflatable pouch extending over less than 75% of the circumference;
the occlusive cuff being operable between
an occluding configuration wherein the inflatable pouch is filled with fluid to exert a compression onto the anatomical duct for occluding the anatomical duct without pinching the anatomical duct, and
a released configuration wherein the inflatable pouch is deflated to release the compression onto the anatomical duct and at least partially open the anatomical duct; and
wherein the band is remote from the housing, the pump being configured to transfer fluid between the fluid container and the inflatable pouch to selectively increase or decrease a volume of fluid in the pouch.

18. The implantable occlusive system of claim 17, consisting of an artificial urinary sphincter.
